(19) United States
(12) Patent Application Publication
(10) Pub. No.: US 2009/0012136 A1
(43) Pub. Date: Jan. 8, 2009

(54) UNIT DOSE FORMULATIONS AND METHODS OF TREATING AND PREVENTING THROMBOSIS WITH THROMBOXANE RECEPTOR ANTAGONISTS

(75) Inventors: Gillian Stephens, San Francisco, CA (US); Dacao Gao, Dana Point, CA (US); Patrick Andre, San Mateo, CA (US); David R. Phillips, San Mateo, CA (US)

Correspondence Address: SEED INTELLECTUAL PROPERTY LAW GROUP PLLC, 701 FIFTH AVE, SUITE 5400, SEATTLE, WA, 98104, US

(73) Assignee: PORTOLA PHARMACEUTICALS, INC., South San Francisco, CA (US)

(21) Appl. No.: 12/114,646

(22) Filed: May 2, 2008

Related U.S. Application Data
(60) Provisional application No. 60/915,784, filed on May 3, 2007.
(60) Provisional application No. 60/915,785, filed on May 3, 2007.
(60) Provisional application No. 60/947,316, filed on Jun. 29, 2007.
(60) Provisional application No. 60/947,289, filed on Jun. 29, 2007.

(51) Int. Cl. A61K 31/421; C12N 5/00; C12Q 1/02; A61P 7/02

(52) U.S. Cl. 514/374; 435/375; 435/29

(57) ABSTRACT

The present invention provides new methods of treating thrombosis and cardiovascular diseases using of antithrombotic agents, as well as methods of determining therapeutically effective amounts of antithrombotic agents and unit dose formulations thereof.

### CROSS-REFERENCES TO RELATED APPLICATIONS

This application claims the benefit under 35 U.S.C. § 119(e) of U.S. Provisional Patent Application No. 60/915,784, filed May 3, 2007; U.S. Provisional Patent Application No. 60/915,785, filed May 3, 2007; U.S. Provisional Patent Application No. 60/947,316, filed Jun. 29, 2007; and U.S. Provisional Patent Application No. 60/947,289, filed Jun. 29, 2007, where these (four) provisional applications are incorporated herein by reference in their entireties.

### BACKGROUND

1. Technical Field

The present invention relates to methods of treating or preventing thrombosis and cardiovascular diseases and disorders using antithrombotic agents such as thromboxane receptor antagonists, as well as unit dose formulations thereof.

2. Description of the Related Art

Arterial thrombosis causes acute myocardial infarction and thrombotic stroke and is a major contributor to morbidity and mortality in the Western world. The role of platelets in arterial thrombosis is well established as arterial thrombi are composed primarily of platelets, and antiplatelet drugs are effective in reducing the incidence of acute myocardial infarction and thrombotic stroke. Platelets play a pivotal role not only in the formation of arterial thrombosis but also in the progression of atherosclerotic disease itself.

Platelet involvement in the progression of atherosclerosis is a more recent finding that evolved from the recognition that atherosclerotic disease is a response to inflammation and that inflammatory mediators released from platelet thrombi (e.g., sCD40L, RANTES, TGFα, PF4, PDGF) are potent contributors to the development of atherosclerotic lesions (see, Huo Y., et al., Nat Med 9:61-67 (2003); Massberg S., et al., J. Exp. Med. 196:887-896 (2002); Burger P. C., et al., Blood 101:2661-2666 (2003)).

Mechanisms responsible for platelet thrombosis have been identified. Platelet adhesion under arterial shear rates is mediated primarily by collagen, which recruits von Willebrand factor from plasma, which in turn is recognized by platelet membrane GP Ib-V-IX, triggering the recruitment of the platelets at the site of vascular injury. Platelets also bind directly to collagen via 2 collagen receptors on platelets, the integrin α₂β₁, and the immunoglobulin-containing collagen receptor, GP VI.

Platelet activation is initially mediated by the primary agonists; collagen during platelet adhesion, and thrombin, a protease generated in response to tissue factor (TF) exposed at sites of vascular lesions and also by the engagement of GP IIb-IIIa by different ligands (fibrinogen, vWF, CD40L). In addition, several secondary agonists released from activated platelets function in autocrine loops to potentiate platelet activation. One is thromboxane A₂ (TXA₂), a product of the prostanoid pathway that is initiated by the release of arachidonic acid from phospholipids in response to the primary platelet agonists. Released arachidonate is sequentially modified by COX-1, a platelet enzyme, yielding PGH₂, a substrate for the widely distributed thromboxane synthase, which produces thromboxane A₂ (TXA₂). Both products, PGH₂ and TXA₂ are potent platelet agonists that induce platelet activation by binding to the TXA₂ receptor, also known as TP. Another secondary agonist is ADP, which is released from platelet dense bodies upon platelet activation. ADP binds to two G-protein coupled receptors, P₂Y₁ and P₂Y₁₂. Additional secondary mediators include Gas6 and CD40L.

Platelet activation is characterized by shape change, induction of fibrinogen receptor expression, and release of granular contents, leading to aggregation and plug formation. While this response is essential for hemostasis, it is also important in the pathogenesis of a broad spectrum of thrombosis-related diseases, including myocardial infarction, stroke, and unstable angina.

The primary antiplatelet drug used for regulation of platelet function in patients with cardiovascular disease is aspirin. The extensive use of aspirin is based on hundreds of randomized clinical trials, which show a reduction in adverse events by 20-25% (BMJ 324:71-86 (2002)). Although the success of aspirin is remarkable, it has become apparent that some individuals do not benefit from aspirin therapy. Certain patients are resistant to aspirin and suffer thrombotic events despite aspirin therapy. In addition, some patients at risk of cardiovascular thrombotic events are aspirin sensitive and cannot avail themselves of the cardiovascular protection provided by aspirin.

Given that a substantial number of patients that might otherwise benefit from aspirin therapy are resistant or intolerant to aspirin, alternative drugs that mimic the cardiovascular protection provided by aspirin, but do not initiate the inflammatory reactions inherent to aspirin, are sought. One class of drugs being developed is anti-thrombotic agents that inhibit thromboxane (TXA₂)-mediated platelet aggregation. TXA₂, the prothrombotic product resulting from the action of COX-1, activates platelets by acting on the TXA₂ receptor, also known as TP, and sometimes referred to as the TP receptor (TP) TXA₂ is the prothrombotic mediator blocked by aspirin, and TP antagonism provides an alternative strategy for blocking the action of this prothrombotic mediator.

The discovery and development of TXA₂ receptor antagonists has been an objective of many pharmaceutical companies for approximately 30 years (see, Dogne J-M, et al., Exp. Opin. Ther. Patents 11: 1663-1675 (2001)). The compounds identified by these companies, either with or without concominant TXA₂ synthase inhibitory activity, include ifetroban (BMS), ridogrel (Janssen), terbogrel (BI), UK-147535 (Pfizer), GR 32191 (Glaxo), and S-18886 (Servier). Preclinical pharmacology has established that this class of compounds has effective antithrombotic activity obtained by inhibition of the thromboxane pathway. These compounds also prevent vasoconstriction induced by TXA₂ and other prostanoids that act on the TXA₂ receptor within the vascular bed. Unfortunately, however, the Phase II/III trials of TXA₂ antagonists have not proven successful, and none of these compounds have reached the marketplace.

Clearly, there remains a need in the art for identifying additional anti-thrombotic agents and therapeutically effective methods of using antithrombotic agent, including TP antagonists, in order to provide alternative means of treating and preventing thrombosis, including effective treatments for aspirin resistant or aspirin-sensitive patients.

### BRIEF SUMMARY

In one embodiment, the present invention provides a method of inhibiting the aggregation of platelets, comprising contacting platelets with ifetroban at a concentration greater than 100 nM or a concentration greater than or about 350 nM.

In a related embodiment, the present invention provides a method of treating or preventing thrombosis in a patient, comprising administering to the patient an amount of ifetroban sufficient to achieve a plasma concentration greater than 100 nM, or greater than or about 350 nM, for at least 24 hours. In a related embodiment, the present invention provides a method of treating or preventing thrombosis in a patient, comprising administered to the patient an amount of ifetroban sufficient to achieve a steady state trough plasma concentration of greater than 250 nM, or greater than or about 350 nM, for at least 24 hours. In particular embodiments, the amount of ifetroban administered is about 450 mg per day. In particular embodiments, the amount of ifetroban administered is between 1 and 10 mg/kg/day or about 6 or 7 mg/kg/day. In another particular embodiment, the amount of ifetroban is sufficient to achieve a plasma concentration greater than or about 250 nM or 350 nM for at least 24 hours. In one embodiment, the amount of ifetroban is between 6 and 10 mg/kg/day. In another embodiment, it is between 5 and 10 mg/kg/day.

In other related embodiments, the present invention includes a method of treating or preventing thrombosis in a patient, comprising administering to the patient an amount of ifetroban sufficient to achieve a steady-state blood plasma concentration in the range of 350 nM and 1000 nM for some time.

In other related embodiments, the present invention includes a method of treating or preventing thrombosis in a patient, comprising administering to the patient an amount of ifetroban sufficient to achieve a blood plasma concentration having a Cmax in the range of 1500 to 2500 ng/mL.

In other related embodiments, the present invention includes a method of treating or preventing thrombosis in a patient, comprising administering to the patient an amount of ifetroban sufficient to achieve a total blood plasma concentration having a mean trough concentration of about 154 ng/mL.

In other related embodiments, the present invention includes a method of treating or preventing thrombosis in a patient, comprising administering to the patient an amount of ifetroban sufficient to achieve a total blood plasma concentration having a peak to trough concentration ratio of 15 or less.

In a further related embodiment, the present invention provides a method of treating or preventing thrombosis in a patient, comprising administering a therapeutically effective plasma concentration of an antithrombotic agent to the patient, wherein the therapeutically effective plasma concentration is determined by a method comprising: contacting a blood sample obtained from a mammal with a physiological platelet agonist in an amount sufficient to induce platelet aggregation in the blood sample and measuring a first amount of platelet aggregation; and subsequently contacting the blood sample with a plasma concentration of an antithrombotic agent and measuring a second amount of platelet aggregation in the blood sample, wherein if the second amount of platelet aggregation is at least 25% lower than the first amount of platelet aggregation, the plasma concentration of the antithrombotic agent is a therapeutically effective plasma concentration. In one embodiment, the physiological platelet agonist is collagen. In another embodiment, the method further comprises anticoagulating the blood sample prior to contacting the blood sample with the physiological platelet agonist. In a further embodiment, the antithrombotic agent is a thromboxane receptor antagonist. In a particular embodiment, the thromboxane receptor antagonist is ifetroban. In certain embodiments, the first amount and second amount of platelet aggregation is measured by light transmittance aggregometry. In certain embodiments, the first amount and second amount of platelet aggregation is measured using a real time perfusion chamber.

In yet a further embodiment, the present invention provides a unit dose formulation of ifetroban, comprising a pharmaceutically acceptable carrier and an amount of ifetroban sufficient to maintain a plasma concentration of at least 250 nM or at least 350 nM for at least 24 hours. In one embodiment, the formulation is adapted for once a day administration and the amount of ifetroban is a dose between 6-10 mg/kg. In another embodiment, the formulation is adapted for twice a day administration and the amount of ifetroban is a dose between 3-5 mg/kg.

Another embodiment of the present invention provides a method for determining an effective concentration of an antithrombotic agent for inhibiting aggregation of mammalian platelets, comprising: contacting a blood sample obtained from a mammal with a physiological platelet agonist in an amount sufficient to induce platelet aggregation in the blood sample and measuring a first amount of platelet aggregation; and subsequently contacting the blood sample with a plasma concentration of the antithrombotic agent and measuring a second amount of platelet aggregation in the blood sample, wherein, if the second amount of platelet aggregation is at least 25% lower than the first amount of platelet aggregation, the plasma concentration is an effective concentration of the antithrombotic agent for inhibiting aggregation of mammalian platelets. In particular embodiments, the physiological platelet agonist is collagen, epinephrine, or ADP. In certain embodiments, the method further comprises anticoagulating the blood sample prior to contacting the blood sample with the physiological platelet agonist. In particular embodiments, the antithrombotic agent is a thromboxane receptor antagonist.

In one particular embodiment, the present invention includes a method for inhibiting platelet aggregation in a patient in need thereof, comprising administering to a patient in need thereof a therapeutic concentration of an antithrombotic agent, wherein said therapeutic concentration is determined by a method comprising: contacting a blood sample obtained from a mammal with a physiological platelet agonist in an amount sufficient to induce platelet aggregation in the blood sample and measuring a first amount of platelet aggregation; and subsequently contacting the blood sample with a plasma concentration of an antithrombotic agent and measuring a second amount of platelet aggregation in the blood sample, wherein if the second amount of platelet aggregation is at least 25% lower than the first amount of platelet aggregation, the plasma concentration of the antithrombotic agent is a therapeutically effective plasma concentration.

In particular embodiments, methods and compositions of the present invention are used to treat or prevent a cardiovascular disease or disorder, including, e.g., myocardial infarction, thrombotic stroke, atherosclerotic disease, unstable angina, refractory angina, transient ischemic attacks, embolic stroke, disseminated intravascular coagulation, septic shock, deep venous thrombosis, pulmonary embolism, reocclusion, restenosis, pulmonary embolism, and occlusive coronary thrombus or other complications resulting from thrombolytic therapy, percutaneous transluminal coronary angioplasty, or coronary artery bypass grafts. In addition, the methods and compositions of the present invention may be used to treat or prevent pulmonary hypertension, e.g., hypoxia-induced pulmonary hypertension, and intravascular thrombosis

### BRIEF DESCRIPTION OF THE SEVERAL VIEWS OF THE DRAWING(S)

FIG. 1 is a schematic diagram depicting the mechanisms leading to platelet aggregation in response to collagen stimulation.

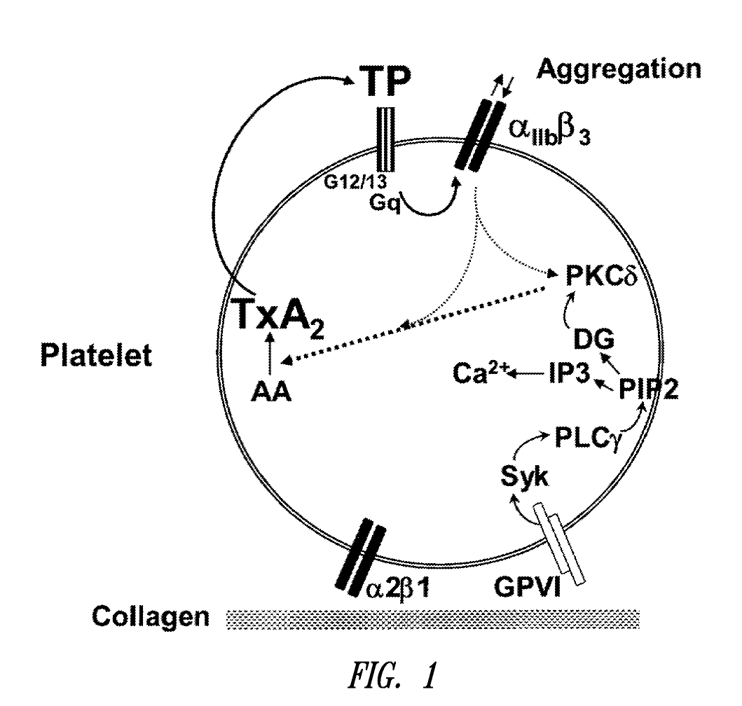

FIG. 2 is a graph showing dose-responsive ifetroban inhibition of U-46619 and collagen-induced platelet aggregation as determined by light transmittance aggregometry (LTA). The inhibition of collagen-induced platelet aggregation by chronic aspirin treatment is indicated by the vertical bar.

FIG. 3 is a graph showing mean thrombotic profiles over time in response to the indicated dosages of ifetroban or aspirin. The data are expressed as mean±sem of thrombus size over time.

FIG. 4 provides graphs depicting the antithrombotic activity of 100 nm or 350 nm ifetroban (103) versus aspirin in healthy volunteers (FIG. 4A) and aspirin-intolerant (AERD)-asthmatic patients (FIG. 4B) as determined using a perfusion chamber assay. The inhibition of thrombosis is shown as a reduction in the fluorescence intensity as compared to controls.

FIG. 5 provides bar graphs showing the antithrombotic activity of 1 μM, 100 nM, and 350 nM ifetroban (103) versus aspiring in healthy volunteers (FIG. 5A) and aspirin-intolerant (AERD)-asthmatic patients (FIG. 5B) as determined using a collagen-induced platelet aggregation assay. As shown, a statistically significant inhibition of collagen-induced platelet aggregation was shown at concentrations >100 nM in both normal volunteers (P=0.0281) and AERD patients. NS indicates not significant.

FIG. 6 is a graph showing the antithrombotic activity of 2 mM SQ29548 and 5 μM terbogrel as compared to aspirin in an aspirin-intolerant (AERD)-asthmatic patient, as determined using a perfusion chamber assay.

FIG. 7 provides graphs depicting the antithrombotic activity of 1 μM, 350 nM, and 100 nM ifetroban versus aspirin in healthy volunteers (FIG. 7A) and aspirin-intolerant (AERD-asthmatic patients (FIG. 7B) as determined by an arachidonic acid-induced platelet aggregation assay.

### DETAILED DESCRIPTION

The present invention is based upon the surprising discovery that previously used in vitro assays of platelet aggregation substantially underestimate the amount of antithrombotic agent required to achieve a therapeutic benefit in vivo. For example, during the development of thromboxane receptor (TP) antagonists, the pharmacodynamic assay of choice utilized to monitor activity of TP antagonists in humans was the measurement of the inhibition of platelet shape change and platelet aggregation induced by U-46619, a TXA₂ mimetic. Although U-46619 directly stimulates platelets by binding to TP, the present invention discovered that inhibition of U-46619-induced platelet shape change or aggregation is not predictive of the inhibition of thrombosis in humans. None of the clinical trials performed with thromboxane (TX) synthase, TP or mixed TX synthase/TP inhibitors used collagen-induced platelet aggregation or perfusion chamber as pharmacodynamic assays for dose selection or drug monitoring in their clinical development programs. Therefore, it was a surprising and unexpected finding of the present invention that TP receptors are differentially involved in platelet aggregation induced by physiological platelet agonists such as collagen as compared to U-46619.

In one embodiment, the present invention establishes appropriate assays, utilizing physiological platelet agonists, for determining concentrations of antithrombotic agents, including TP antagonists such as ifetroban, that inhibit platelet aggregation, e.g., to the same extent as aspirin. In particular embodiments, the determined concentration provides a clinical benefit comparable to aspirin. Accordingly, the present invention provides new methods of identifying antithrombotic agents and method of determining therapeutically effective dosages of antithrombotic agents. In addition, the present invention includes methods of treating or preventing thrombosis and cardiovascular diseases and disorders, comprising administering to a patient an antithrombotic agent, such as a TP antagonist, in a dosage substantially greater than those previously used. Additional related methods are directed at administering to a patient an amount of an antithrombotic agent sufficient to achieve and/or maintain a plasma concentration substantially higher than previously understood to be required for therapeutic benefit.

The practice of the present invention will employ, unless indicated specifically to the contrary, conventional methods of chemistry, virology, immunology, microbiology, cell biology, pharmacology, molecular biology and recombinant DNA techniques within the skill of the art, many of which are described below for the purpose of illustration. Such techniques are explained fully in the literature.

As used in this specification and the appended claims, the singular forms “a,” “an” and “the” include plural references unless the content clearly dictates otherwise.

### A. Methods of Identifying and Determining Therapeutically Effective Dosages of Antithrombotic Agents

The present invention provides methods of identifying antithrombotic agents and related methods of determining a therapeutically effective amount of an antithrombotic agent. These methods are based upon the discovery that previous in vitro assays of antithrombotic activity that utilize the TXA₂ mimetic, U-46619, to induce platelet shape change or aggregation do not accurately predict the effective amount of antithrombotic agent required in vivo. Therefore, the presently claimed methods utilize physiological platelet agonists, such as collagen, epinephrine and ADP, to induce platelet aggregation. The methods include all types of assays previously practiced using U-46619 or other non-physiological platelet agonists, wherein a physiological platelet agonist is substituted for the non-physiological platelet agonist.

In general, methods of the present invention comprise contacting platelets in solution with a physiological platelet agonist in the absence or presence of an amount of or concentration of a candidate antithrombotic agent and determining whether the presence of the candidate antithrombotic agent reduces the amount of platelet aggregation induced by the physiological platelet agonist.

Platelet aggregation may be determined based upon any known characteristic of platelet aggregation, such as platelet shape change, induction of fibrinogen receptor expression, release of granular contents, platelet adhesion, and clot formation. Methods of measuring each of these characteristics are known and available in the art. In particular embodiments, platelet aggregation is measured using light transmittance aggregometry (LTA) or perfusion chambers. In particular embodiments, platelets are fluorescently labeled.

Physiological platelet agonists include endogenous molecules that stimulate, induce, or otherwise contribute to platelet aggregation in vivo. Various physiological platelet agonists are known in the art, including but not limited to collagen, e.g., type III collagen, type I collagen, oxidized LDL, thrombospondin, epinephrine, CD40L, thrombin, and adenosine 5′-diphosphate (ADP). Any of these or other physiological platelet agonists may be used alone, or in any combination.

Platelets used for the methods described herein may be obtained from any animal, preferably a mammal such as a human. Platelets in solution include blood and platelet-rich plasma. Blood samples may be readily obtained from an animal, e.g., using a needle and syringe. Platelet-rich plasma may be prepared from blood using routine methods.

In certain embodiments, platelets in solution are anticoagulated using a Factor Xa inhibitor that does not affect physiological calcium levels before or while being exposed to a physiological platelet agonist or antithrombotic agent. Examples of Factor Xa inhibitors that do not affect physiological calcium levels are known in the art. One example of a suitable Factor Xa inhibitor is C921-78 (Andre P., et al. Circulation 2003; 108:2697-2703).

Methods of the present invention may be used to identify an antithrombotic agent by screening candidate agents for their ability to inhibit platelet aggregation induced by a physiological platelet agonist. Candidate agents include, e.g., organic molecules, peptides, polypeptides, antibodies, nanobodies, and derivatives and mimetics thereof. In certain embodiments, a library of candidate antithrombotic agents is screened using methods of the present invention. Candidate agents may be screened individually or in pools, and those agents having activity identified by further dilution.

Methods of the present invention may be used to determine a concentration sufficient to inhibit platelet aggregation, e.g., by testing increasing amounts of a candidate or known antithrombotic agent for their ability to inhibit platelet aggregation induced by a physiological platelet agonist. In certain embodiments, therapeutically effective concentrations of antithrombotic agents are determined using methods of the present invention, since the present invention establishes that there is a correlation between the concentration required to inhibit platelet aggregation induced by a physiological platelet agonist in vitro and the amount required to inhibit platelet aggregation in vivo in a mammalian patient, such as a human.

In various embodiments, a candidate antithrombotic agent is identified as an antithrombotic agent if it reduces platelet aggregation as measured by a characteristic or assay described herein by at least 10%, 15%, 20%, 25%, 30%, 40%, 50%, 60%, 70%, 80%, 90%, or 100%, as compared to a negative control. In other embodiments, an effective concentration is defined as the concentration of an antithrombotic agent required to reduce platelet aggregation as measured by an characteristic or assay described herein by at least 10%, 15%, 20%, 25%, 30%, 40%, 50%, 60%, 70%, 80%, 90%, or 100%, as compared to a negative control.

In other embodiments, a candidate antithrombotic agent is identified as an antithrombotic agent, if it reduces or inhibits platelet aggregation by an amount equal to or greater than the amount reduced by aspirin. In a related embodiment, an effective concentration of a candidate antithrombotic agent is identified as such, if it reduces or inhibits platelet aggregation by an amount equal to or greater than the amount reduced by aspirin. In one embodiment, the amount of aggregation inhibited by aspirin is determined using blood or platelet-rich plasma obtained from a mammal that has been administered aspirin for at least three days. In one embodiment, the animal is a human that has been administered aspirin at approximately 325 mg/day for at least one or two weeks.

Thus, in certain embodiments, the present invention includes a method for determining whether a candidate antithrombotic agent is an antithrombotic agent, comprising contacting a blood or serum sample obtained from a mammal with a physiological platelet agonist in an amount sufficient to induce platelet aggregation in the blood or serum sample and measuring a first amount of platelet aggregation, and subsequently contacting the blood or serum sample with a plasma concentration of the candidate antithrombotic agent and measuring a second amount of platelet aggregation in the blood or serum sample, wherein, if the second amount of platelet aggregation is at least 25% lower than the first amount of platelet aggregation, the candidate antithrombotic agent is an antithrombotic agent.

In related embodiments, the present invention includes a method for determining if a candidate antithrombotic agent is an antithrombotic agent, comprising: (1) contacting a blood or serum sample obtained from a mammal with a physiological platelet agonist in an amount sufficient to induce platelet aggregation in the blood or serum sample, and measuring a first amount of platelet aggregation; and (2) contacting a comparable blood or serum sample obtained from a mammal with a physiological platelet agonist in an amount sufficient to induce platelet aggregation in the blood or serum sample in the presence of an amount of a candidate antithrombotic agent, and measuring a second amount of platelet aggregation, wherein, if the second amount of platelet aggregation is at least 25% lower than the first amount of platelet aggregation, the candidate antithrombotic agent is identified as an antithrombotic agent.

In other related embodiments, the present invention includes a method for determining an effective concentration of an antithrombotic agent for inhibiting aggregation of mammalian platelets, comprising contacting a blood or serum sample obtained from a mammal with a physiological platelet agonist in an amount sufficient to induce platelet aggregation in the blood or serum sample and measuring a first amount of platelet aggregation, and subsequently contacting the blood or serum sample with a plasma concentration of the antithrombotic agent and measuring a second amount of platelet aggregation in the blood or serum sample, wherein, if the second amount of platelet aggregation is at least 25% lower than the first amount of platelet aggregation, the concentration is an effective concentration of the antithrombotic agent for inhibiting aggregation of mammalian platelets.

In additional related embodiments, the present invention includes a method for determining an effective concentration of an antithrombotic agent for inhibiting aggregation of mammalian platelets, comprising (1) contacting a blood or serum sample obtained from a mammal with a physiological platelet agonist in an amount sufficient to induce platelet aggregation in the blood or serum sample, and measuring a first amount of platelet aggregation; and (2) contacting a comparable blood or serum sample obtained from a mammal with a physiological platelet agonist in an amount sufficient to induce platelet aggregation in the blood or serum sample in the presence of an amount of an antithrombotic agent, and measuring a second amount of platelet aggregation, wherein, if the second amount of platelet aggregation is at least 25% lower than the first amount of platelet aggregation, the concentration is an effective concentration of the antithrombotic agent for inhibiting aggregation of mammalian platelets.

In one embodiment, a method of the invention is practiced by performing light transmittance aggregometry on a sample of blood or platelet-rich plasma anticoagulated with a Factor Xa inhibitor and induced to aggregate using a physiological platelet agonist, such as collagen (e.g., 1, 2, 3, 4, 5, 6, 7, 8, 9, or 10 mg/ml) and, in certain assays, exposed to an antithrombotic agent.

In another embodiment, a method of the invention is practiced by performing a real time perfusion assay on a sample of blood or platelet-rich plasma anticoagulated with a Factor Xa inhibitor and induced to aggregate using a physiological platelet agonist, such as collagen (e.g., 1, 2, 3, 4, 5, 6, 7, 8, 9, or 10 mg/ml) and, in certain assays, exposed to an antithrombotic agent. Perfusion chambers were developed more than 30 years ago to study platelet thrombosis in samples of non-anticoagulated or anticoagulated blood exposed to physiological thrombogenic surfaces under defined conditions of shear, such as those encountered in moderately stenosed coronary arteries (e.g., 1600/sec). These techniques and others that may be utilized according to the present invention are described in Sakariassen, K. S. et al., J. Thromb. Haemost. 2:1681-90 (2004) and Sakariassen, K. S. et al., Thromb. Res. 104:149-74 (2001).

Methods of the present invention may be readily adapted for monitoring drug activity, e.g., in clinical trials. Blood samples are obtained from a patient treated with an antithrombotic agent. In particular embodiments, platelets in solution (e.g., blood) are fluorescently labeled, and the kinetics of thrombosis may be observed in real-time. For example, whole blood anticoagulated with a Factor Xa inhibitor is exposed in a perfusion chamber to a physiological platelet agonist such as type III collagen under defined rates of shear, such as those encountered in stenosed coronary arteries. Analysis of thrombotic deposits in the perfusion chamber is performed in real time via computer monitoring of variations in fluorescence intensity. Parameters such as maximum thrombus peak, the time to reach maximum extent of thrombus formation, and rates of thrombus growth and dissolution may be determined. Such assays may be performed using miniaturized devices requiring only limited amounts of blood (approximately six ml of blood per assay, or less). Blood samples may be taken from a patient at one time point or at various time points during treatment with an antithrombotic agent, and the efficacy of the treatments, thus, determined based upon the determined amount of platelet aggregation, e.g., as compared to a negative control or pre-determined desired or control value.

Antithrombotic agents include, but are not limited to, anticoagulants, antiplatelet agents, and thrombolytics. Specific examples of anticoagulants include vitamin K antagonists, unfractionated heparin, and low molecular weight heparins. Specific examples of antiplatelet agents include platelet aggregation inhibitors such as aspirin, ticlopidine, and dipyridamole. Specific examples of thrombolytics include streptokinase, urokinase, and tissue plasminogen activator.

Additional antithrombotic agents include specific inhibitors of targets involved in the coagulation pathway, such as thrombin, Factor Xa, ADP receptor, thromboxane, or thromboxane receptor (TP).

1. TP Antagonists

The term “thromboxane A2 receptor antagonist” or “thromboxane receptor antagonist” or “TP antagonist” as used herein refers to a compound that inhibits the expression or activity of a thromboxane receptor by at least or at least about 30%, 35%, 40%, 45%, 50%, 55%, 60%, 65%, 70%, 75%, 80%, 85%, 90%, 95%, 96%, 97%, 98%, 99%, or 100% in a standard bioassay or in vivo or when used in a therapeutically effective dose. In certain embodiments, a TP antagonist inhibits binding of thromboxane A₂ to TP. TP antagonists include competitive antagonists (i.e., antagonists that compete with an agonist for TP) and non-competitive antagonists. TP antagonists include antibodies to the receptor. The antibodies may be monoclonal. They may be human or humanized antibodies. TP antagonists also include thromboxane synthase inhibitors, as well as compounds that have both TP antagonist activity and thromboxane synthase inhibitor activity.

TP antagonists include, for example, small molecules such as ifetroban (BMS; [1S-(1α,2α,3α,4α)]-2-[[3-[4-[(pentylamino)carbonyl]-2-oxazolyl]-7-oxabicyclo[2.2.1]hept-2-yl]methyl]benzenepropanoic acid), 5-hexenoic acid, 6-[3-[[(cyanoamino)[(1,1-dimethylethyl)amino]methylene]amino]phenyl]-6-(3-pyridinyl)-, (ε-) (terbogrel), 5-[(2-chlorophenyl)methyl]-4,5,6,7-tetrahydrothieno[3,2-c]pyridine, N-[2-(methylthio)ethyl]-2-[(3,3,3-trifluoropropyl)thio]-5′-adenylic acid, monoanhydride with dichloromethylenebisphosphonic acid, 2-(propylthio)-5′-adenylic acid, monoanhydride with dichloromethylene bis(phosphonic acid), methyl(+)-(S)-α-(2-chlorophenyl)-6,7-dihydrothieno[3,2-c]pyridine-5(4H)-acetate, 2-acetoxy-5-(α-cyclopropylcarbonyl-2-fluorobenzyl)-4,5,6,7-tetrahydrothieno[3,2-c]pyridine, 4-methoxy-N,N′-bis(3-pyridinylmethyl)-1,3-benzenedicarboxamide (picotamide), ridogrel (Janssen), sulotroban, UK-147535 (Pfizer), GR 32191 (Glaxo), variprost, and S-18886 (Servier).

Additional TP antagonists suitable for use herein are also described in U.S. Pat. No. 6,509,348. These include, but are not limited to, the interphenylene 7-oxabicycloheptyl substituted heterocyclic amide prostaglandin analogs as disclosed in U.S. Pat. No. 5,100,889, issued Mar. 31, 1992, including [1S-(1α,2α,3α,4α)]-2-[[3-[4-[[(4-cyclohexylbutyl)amino]carbonyl]-2-oxazolyl]-7-oxabicyclo[2.2.1]hept-2-yl]methyl]benzenepropanoic acid (SQ 33,961) which is preferred, or esters or salts thereof; [1S-(1α,2α,3α,4α)]-2-[[3-[4-[[[(4-chlorophenyl)butyl]amino]carbonyl]-2-oxazolyl]-7-oxabicyclo[2.2.1]hept-2-yl]methyl]benzenepropanoic acid or esters, or salts thereof; [1S-((1α,2α,3α,4α)]-3-[[3-[4-[[(4-cyclohexylbutyl)amino]carbonyl]-2-oxazolyl]-7-oxabicyclo[2.2.1]hept-2-yl]methyl]benzeneacetic acid, or esters or salts thereof; [1S-((1α,2α,3α,4α)]-[2-[[3-[4-[[(4-cyclohexylbutyl)amino]carbonyl]-2-oxazolyl]-7-oxabicyclo[2.2.1]hept-2-yl]methyl]phenoxy]acetic acid, or esters or salts thereof; [1S-((1α,2α,3α,4α)]-2-[[3-[4-[[-7,7-dimethyloctyl)amino]carbonyl]-2-oxazolyl]-7-oxabicyclo[2.2.1]hept-2yl]methyl]benzenepropanoic acid, or esters or salts thereof and ifetroban; 7-oxabicycloheptyl substituted heterocyclic amide prostaglandin analogs as disclosed in U.S. Pat. No. 5,100,889, issued Mar. 31, 1992, including [1S-[1α,2α(Z),3α,4α)]-6-[3-[4-[[(4-cyclohexylbutyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[(4-cyclohexylbutyl)amino]carbonyl]-2-thiazolyl]-7-oxabicyclo[2.2.1]hept-2-yl]-4-hexenoic acid, or esters or salts thereof; [1S-[1α,2α(Z),3α,4α)]]-6-[3-[4-[[(4-cyclohexylbutyl)methylamino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(1-pyrrolidinyl)carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(cyclohexylamino)carbonyl]-2-oxazolyl]-7-oxabicyclo[2.2.1]hept-2-yl-4-hexenoic acid or esters or salts thereof; [1S-[1α,2α(Z),3α,4α)]]-6-[3-[4-[[(2-cyclohexylethyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[[2-(4-chloro-phenyl)ethyl]amino]carbonyl]-2-oxazolyl]-7-oxabicyclo-[2.2.1]hept-2-yl]-4-hexenoic acid, or esters or salts thereof; [1S-[1α,2α(Z),3α,4α)]]-6-[3-[4-[[(4-chlorophenyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[[4-(4-chlorophenyl)butyl]amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4a-[[(6-cyclohexylhexyl)amino]carbonyl]-2-oxazolyl]-7-oxabicyclo[2.2.1]hept-2-yl]-4-hexenoic acid, or esters, or salts thereof; [1S-[1α,2α(Z),3α,4α)]]-6-[3-[4-[[(6-cyclohexylhexyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(propylamino)carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[(4-butylphenyl)amino]carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[(2,3-dihydro-1H-indol-1-yl)carbonyl]-2-oxazolyl]-7-oxabicyclo[2.2.1]hept-2-yl]-4-hexenoic acid, or esters or salts thereof; [1S-[1α,2α(Z),3α,4α)]]-6-[3-[4-[[(4-cyclohexylbutyl)amino]carbonyl]-2-oxazolyl]-7-oxabicyclo[2.2.1]hept-2-yl]-N-(phenylsulfonyl)-4-hexenamide; [1S-[1α,2α(Z),3α,4α)]]-6-[3-[4-[[(4-cyclohexylbutyl)amino]carbonyl]-2-oxazolyl]-N-(methylsulfonyl)-7-oxabicyclo[2.2.1]hept-2-yl]-4-hexenamide; [1S-[1α,2α(Z),3α,4α)]]-7-[3-[4-[[(4-cyclohexylbutyl)amino]carbonyl]-2-oxazolyl]-7-oxabicyclo[2.2.1]hept-2-yl]-5-heptenoic acid, or esters or salts thereof; [1S-[1α,2α(Z),3α,4α)]]-6-[3-[4-[[(4-cyclohexylbutyl)amino]carbonyl]-1H-imidazol-2-yl]-7-oxabicyclo-[2.2.1]hept-2-yl]-4-hexenoic acid or esters or salts thereof; [1S-[1α,2α,3α,4α)]-6-[3-[4-[[(7,7-dimethyloctyl)amino]carbonyl]-2-oxazolyl]-7-oxabicyclo[2.2.1]hept-2-yl]-4-hexenoic acid, or esters or salts thereof; [1S-[1α,2α(E),3α,4α)]]-6-[3-[4-[[(4-cyclohexylbutyl)amino]carbonyl]-2-oxazolyl]-7-oxabicyclo[2.2.1]hept-2-yl]-4-hexenoic acid; [1S-[1α,2α,3α,4α)]-3-[4-[[(4-(cyclohexylbutyl)amino]carbonyl]-2-oxazolyl]-7-oxabicyclo[2.2.1]heptane-2-hexanoic acid or esters or salts thereof, with a preferred compound being [1S-[1α,2α(Z),3α,4α)]]-6-[3-[4-[[(4-cyclohexylbutyl)amino]carbonyl]-2-oxazolyl]-7-oxabicyclo-[2.2.1]hept-2-yl]-4-hexenoic acid, or esters or salts thereof; 7-oxabicycloheptane and 7-oxabicycloheptene compounds disclosed in U.S. Pat. No. 4,537,981 to Snitman et al., especially [1S-[1α,2α(Z),3α(1E,3S*,4R*),4α)]]-7-[3-(3-hydroxy-4-phenyl-1-pentenyl)-7-oxabicyclo[2.2.1]hept-2-yl]-5-heptenoic acid (SQ 29,548); the 7-oxabicycloheptane substituted amino prostaglandin analogs disclosed in U.S. Pat. No. 4,416,896 to Nakane et al., especially, [1S-[1α,2α(Z),3α,4α)]]-7-[3-[[2-(phenylamino)carbonyl]hydrazino]methyl]-7-oxabicyclo[2.2.1]hept-2-yl]-5-heptenoic acid; the 7-oxabicycloheptane substituted diamide prostaglandin analogs disclosed in U.S. Pat. No. 4,663,336 to Nakane et al., especially, [1S-[1α,2α(Z),3α,4α)]]-7-[3-[[[[(1-oxoheptyl)amino]acetyl]amino]methyl]-7-oxabicyclo[2.2.1]hept-2-yl]-5-heptenoic acid and the corresponding tetrazole, and [1S-[1α,2α(Z),3α,4α)]]-7-[3-[[[[(4-cyclohexyl-1-oxobutyl)amino]acetyl]amino]methyl]-7-oxabicyclo]2.2.1]hept-2-yl]-5-heptenoic acid; 7-oxabicycloheptane imidazole prostaglandin analogs as disclosed in U.S. Pat. No. 4,977,174, issued Dec. 11, 1990, including [1S-[1α,2α(Z),3α,4α)]]-6-[3-[[4-(4-cyclohexyl-1-hydroxybutyl)-1H-imidazole-1-yl]methyl]-7-oxabicyclo[2.2.1]hept-2-yl]-4-hexenoic acid or its methyl ester; [1S-[1α,2α(Z),3α,4α)]]-6-[3-[[4-(3-cyclohexylpropyl)-1H-imidazol-1-yl]methyl]-7-oxabicyclo[2.2.1]hept-2-yl]-4-hexenoic acid or its methyl ester; [1S-[1α,2α(Z),3α,4α)]]-6-[3-[[4-(4-cyclohexyl-1-oxobutyl)-1H-imidazol-1-yl]methyl]-7-oxabicyclo[2.2.1]hept-2-yl]-4-hexenoic acid or its methyl ester; [1S-[1α,2α(Z),3α,4α)]]-6-[3-(1H-imidazol-1-ylmethyl)-7-oxabicyclo[2.2.1]hept-2-yl]-4-hexenoic acid or its methyl ester; or [1S-[1α,2αZ),3α,4α)]]-6-[3-[[4-[[(4-cyclohexylbutyl)amino]carbonyl]-1H-imidazol-1-yl]methyl-7-oxabicyclo-[2.2.1]hept-2-yl]-4-hexenoic acid, or its methyl ester; the phenoxyalkyl carboxylic acids disclosed in U.S. Pat. No. 4,258,058 to Witte et al., especially 4-[2-(benzenesulfamido)ethyl]phenoxyacetic acid (BM 13,177—Boehringer Mannheim), the sulphonamidophenyl carboxylic acids disclosed in U.S. Pat. No. 4,443,477 to Witte et al., especially 4-[2-(4-chlorobenzenesulfonamido)ethyl]phenylacetic acid (BM 13,505, Boehringer Mannheim), the arylthioalkylphenyl carboxylic acids disclosed in U.S. Pat. No. 4,752,616, especially 4-(3-((4-chlorophenyl)sulfonyl)propyl)benzeneacetic acid. yapiprost, (E)-5-[[[(pyridinyl)[3-(trifluoromethyl)phenyl]methylene]amino]oxy]pentanoic acid also referred to as R68,070—Janssen Research Laboratories, 3-[1-(4-chlorophenylmethyl)-5-fluoro-3-methylindol-2-yl]-2,2-dimethylpropanoic acid [(L-655240 Merck-Frosst) Eur. J. Pharmacol. 135(2):193, Mar. 17, 1987], 5(Z)-7-([2,4,5-cis]-4-(2-hydroxyphenyl)-2-trifluoromethyl-1,3-dioxan-5-yl) heptenoic acid (ICI 185282, Brit. J. Pharmacol. 90 (Proc. Suppl):228 P-Abs, March 87), 5(Z)-7-[2,2-dimethyl-4-phenyl-1,3-dioxan-cis-5-yl]heptenoic acid (ICI 159995, Brit. J. Pharmacol. 86 (Proc. Suppl):808 P-Abs., December 85), N,N′-bis[7-(3-chlorobenzeneaminosulfonyl)-1,2,3,4-tetrahydro-isoquinolyl]disulfonylimide (SKF 88046, Pharmacologist 25(3):116 Abs., 117 Abs, August 83), [1α(Z)-2β,5α]-(+)-7-[5-[[(1,1′-biphenyl)-4-yl]methoxy]-2-(4-morpholinyl)-3-oxocyclopentyl]-4-heptenoic acid (AH 23848-Glaxo, Circulation 72(6):1208, December 85, levallorphan allyl bromide (CM 32,191 Sanofi, Life Sci. 31 (20-21):2261, Nov. 15, 1982), (Z,2-endo-3-oxo)-7-(3-acetyl-2-bicyclo[2.2.1]heptyl-5-hepta-3Z-enoic acid, 4-phenyl-thiosemicarbazone (EP092—Univ. Edinburgh, Brit. J. Pharmacol. 84(3):595, March 85); GR 32,191 (Vapiprost)—[1R-[1α(Z),2β,3β,5α]]-(+)-7-[5-([1,1′-biphenyl]-4-ylmethoxy)-3-hydroxy-2-(1-piperidinyl)cyclopentyl]-4-heptenoic acid; ICI 192,605—4(Z)-6-[(2,4,5-cis)₂-(2-chlorophenyl)-4-(2-hydroxyphenyl)-1,3-dioxan-5-yl]hexenoic acid; BAY u 3405 (ramatroban)-3-[[(4-fluorophenyl)sulfonyl]amino]-1,2,3,4-tetrahydro-9H-carbazole-9-propanoic acid; or ONO 3708—7-[2α,4α-(-(di-methylmethano)-6β-(2-cyclopentyl-2β-hydroxyacetamido)-1α-cyclohexyl]-5(Z)-heptenoic acid; (±)(5Z)-7-[3-endo-[(phenylsulfonyl)amino]bicyclo[2.2.1]hept-2-exo-yl]-heptenoic acid (S-1452, Shionogi domitroban, Anboxan™); (−)6,8-difluoro-9-p-methylsulfonylbenzyl-1,2,3,4-tetrahydrocarbazol-1-yl-acetic acid (L670596, Merck) and (3-[1-(4-chlorobenzyl)-5-fluoro-3-methyl-indol-2-yl]-2,2-dimethylpropanoic acid (L655240, Merck). TP antagonists that may be used according to the present invention also include benzenealkonic acids and benzenesulfonamide derivatives, typically at 1-1000 mg per unit dose and 1-5000 mg per day.

In one particular embodiment, the TP modulator is ifetroban, which is described above or alternatively described as: 3-[2-[[(1S,4R,5S,6R)-5-[4-(pentylcarbamoyl)-1,3-oxazol-2-yl]-7-oxabicyclo[2.2.1]hept-6-yl]methyl]phenyl]propanoate, or ifetroban sodium, which is sodium 3-[2-[[(1S,4R,5S,6R)-5-[4-(pentylcarbamoyl)-1,3-oxazol-2-yl]-7-oxabicyclo[2.2.1]hept-6-yl]methyl]phenyl]propanoate. As used herein, the term ifetroban includes both ifetroban and ifetroban sodium. The structure of ifetroban is shown in Formula I:

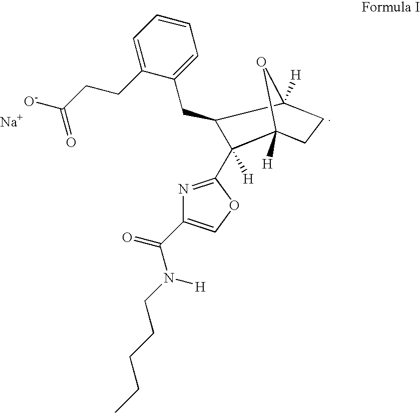

2. ADP Modulators

In particular embodiments, the ADP modulator is an antagonist or inactivator of the platelet ADP receptor, i.e., an ADP receptor antagonist, or a modulator of human CD39 (e.g., recombinant soluble ecto-ADPase/CD39).

The term “ADP receptor antagonist” as used herein refers to a compound that can inhibit or reduce the activity of an ADP receptor by at least about 30%, 40%, 50%, 55%, 60%, 65%, 70%, 75%, 80%, 85%, 90%, 95%, 96%, 97%, 98%, 99%, or 100% when used in therapeutically effective doses or concentrations. ADP receptor antagonists include small molecules and/or prodrugs including thienopyridine derivatives such as, e.g., clopidrogel. ADP receptor antagonists also include polypeptides and nucleic acids that bind to ADP receptors and inhibit their activity. An ADP receptor inactivator is an agent that modifies the receptor so as to block its activity. ADP receptor antagonists can include antibodies to the receptor. The antibodies may be monoclonal. They may be human or humanized antibodies. They may be directed to a human ADP receptor.

Examples of ADP receptor antagonists include, but are not limited to, thienopyridine derivatives such as clopidogrel, prasugrel, and ticlopidine, and direct acting agents such as cangrelor and AZD6140.

Examples of the ADP receptor modulators for use according to the instant invention include: 5-[(2-chlorophenyl)methyl]-4,5,6,7-tetrahydrothieno[3,2-c]pyridine described in U.S. Pat. No. 4,051,141 or U.S. Pat. No. 4,127,580; N-[2-(methylthio)ethyl]-2-[(3,3,3-trifluoropropyl)thio]-5′-adenylic acid, monoanhydride with dichloromethylenebisphosphonic acid described in U.S. Pat. No. 5,955,447 and Journal of Medicinal Chemistry, 1999, Vol. 42, p. 213-220; 2-(propylthio)-5′-adenylic acid, monoanhydride with dichloromethylenebis(phosphonic acid) described in Journal of Medicinal Chemistry, 1999, Vol. 42, p. 213-220; methyl(+)-(S)-α-(2-chlorophenyl)-6,7-dihydrothieno[3,2-c]pyri-dine-5(4H)-acetate described in U.S. Pat. No. 4,529,596, U.S. Pat. No. 4,847,265 or U.S. Pat. Nos. 5,576,328; and 2-acetoxy-5-(α-cyclopropylcarbonyl-2-fluorobenzyl)-4,5,6,7-te-trahydrothieno[3,2-c]pyridine, or pharmaceutically acceptable salts thereof described in U.S. Pat. No. 5,288,726 or WO 02/04461; 5-[(2-chlorophenyl)methyl]-4,5,6,7-tetrahydrothieno[3,2--c]pyridine (particularly, its hydrochloride), N-[2-(methylthio)ethyl]-2-[-(3,3,3-trifluoropropyl)thio]-5′-adenylic acid, monoanhydride with dichloromethylenebisphosphonic acid, methyl(+)-(S)-α-(2-chloropheny-l)-6,7-dihydrothieno[3,2-c]pyridine-5(4H)-acetate (particularly, its sulfate), or 2-acetoxy-5-(α-cyclopropylcarbonyl-2-fluorobenzyl)-4,5-,6,7-tetrahydrothieno[3,2-c]pyridine (particularly, its hydrochloride), or pharmaceutically acceptable salts thereof, more preferably, 2-acetoxy-5-(α-cyclopropylcarbonyl-2-fluorob-enzyl)-4,5,6,7-tetrahydrothieno[3,2-c]pyridine, or pharmaceutically acceptable salts (particularly, its hydrochloride) thereof, and any other ADP modulators or ADP receptor antagonists described in any of these patents and applications.

In particular embodiments, an ADP receptor antagonist according to the present invention is a compound described in U.S. patent application Ser. No. 11/556,490 or a salt thereof. In one embodiment, the ADP receptor antagonist has the structure shown in Formula II:

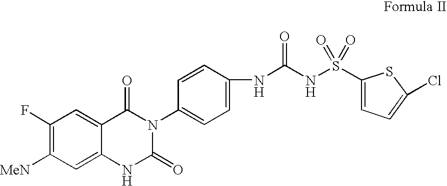

This compound is a reversible inhibitor of ADP-mediated platelet aggregation, which binds specifically to P₂Y₁₂ ADP receptor and has superior pharmacokinetic properties to clopidogrel. In addition, it has been demonstrated to de-aggregate preformed thrombi.

Additional and related antithrombotic agents are described, e.g., in U.S. Pat. Nos. 6,689,786, 7,022,731, 6,906,063, 7,056,926, 6,667,306, 6,762,029, 6,844,367, 6,376,515, 6,835,739, 7,022,695, 6,211,154 6,545,054, 6,777,413, 6,534,535, 6,545,055, 6,638,980, 6,720,317, 6,686,368, 6,632,815, 6,673,817, and 7,022,695, and U.S. patent application Ser. Nos. 11/304,054, 11/107,324, 11/236,051, 10/942,733, 10/959,909, 11/158,274, 11/298,317, 11/298,296, and 11/284,805. These agents may be purchased commercially or manufactured according to published methods.

In particular embodiments, the ADP modulator is an antagonist or inactivator of the platelet ADP receptor or a modulator of human CD39 (e.g., recombinant soluble ecto-ADPase/CD39).

ADP receptor modulators can be easily prepared according to the methods described, e.g., in U.S. Pat. No. 4,051,141, U.S. Pat. No. 4,127,580, U.S. Pat. No. 5,955,447, Journal of Medicinal Chemistry, 1999, Vol. 42, p. 213-220, U.S. Pat. No. 5,721,219, U.S. Pat. No. 4,529,596, U.S. Pat. No. 4,847,265, U.S. Pat. No. 5,576,328, U.S. Pat. No. 5,288,726 or WO 02/04461 or the analogous methods thereto (see also U.S. Patent Application Publication No. 20050192245 which is incorporated herein by reference as to the ADP modulator subject matter disclosed therein).

### B. Methods of Treating Thrombosis and Cardiovascular Diseases Using Antithrombotic Agents

Methods of the present invention may be practiced both in vitro and in vivo to inhibit, reduce, or prevent platelet aggregation or blood coagulation, or to treat or prevent thrombosis and related cardiovascular diseases and disorders. In one embodiment, methods of the present invention are practiced on platelet preparations being stored prior to use. In other embodiments, methods of the present invention are practiced in vivo on patients, which include mammals and, in particular, humans.

Methods of determining an effective or therapeutic concentration of an antithrombotic agent may be used to determine an appropriate dosage or amount to use to treat a patient in need thereof, e.g., to treat or prevent thrombosis in the patient. In one embodiment, the present invention includes a method of inhibiting or preventing platelet aggregation in a patient in need thereof, comprising administering to a patient in need thereof a therapeutic concentration of an antithrombotic agent, wherein said therapeutic concentration is determined by: contacting a blood sample obtained from a mammal with a physiological platelet agonist in an amount sufficient to induce platelet aggregation in the blood sample and measuring a first amount of platelet aggregation; and (b) subsequently contacting the blood sample with a plasma concentration of an antithrombotic agent and measuring a second amount of platelet aggregation in the blood sample, wherein if the second amount of platelet aggregation is at least 25% lower than the first amount of platelet aggregation, the plasma concentration of the antithrombotic agent is a therapeutically effective plasma concentration.

Arterial thrombosis and disorders of coagulation are associated with a variety of cardiovascular-related diseases and disorders, including but not limited to, myocardial infarction, thrombotic stroke, atherosclerotic disease, unstable angina, refractory angina, transient ischemic attacks, embolic stroke, disseminated intravascular coagulation, septic shock, deep venous thrombosis, pulmonary embolism, reocclusion, restenosis, pulmonary embolism, and occlusive coronary thrombus or other complications resulting from thrombolytic therapy, percutaneous transluminal coronary angioplasty, or coronary artery bypass grafts. In addition, the methods and compositions of the present invention may be used to treat or prevent pulmonary hypertension, e.g., hypoxia-induced pulmonary hypertension, and intravascular thrombosis, which have been linked to Cox-2 (Cathcart, M. C. et al., J. Pharmacol. Exp. Ther. Mar. 28, 2008 DOI: 10.1124/jpet.107.134221). Methods of the present invention may be used in the treatment or prevention of any of these and other thrombosis or coagulation-related diseases and disorders.

As used herein, unless the context makes clear otherwise, “treat,” and similar word such as “treatment,” “treating” etc., is an approach for obtaining beneficial or desired results, including and preferably clinical results. Treatment can involve optionally either the reducing or amelioration of a disease or condition, (e.g., thrombosis or a related disease or disorder), or the delaying of the progression of the disease or condition.

As used herein, unless the context makes clear otherwise, “prevent,” and similar word such as “prevention,” “preventing” etc., is an approach for preventing the onset or recurrence of a disease or condition, (e.g., thrombosis or a related disease or disorder) or preventing the occurrence or recurrence of the symptoms of a disease or condition, or optionally an approach for delaying the onset or recurrence of a disease or condition or delaying the occurrence or recurrence of the symptoms of a disease or condition.

Generally, a subject is provided with an effective amount of an antithrombotic agent. As used herein, an “effective amount” or a “therapeutically effective amount” of a substance, e.g., an antithrombotic agent, is that amount sufficient to affect a desired biological or psychological effect, such as beneficial results, including clinical results. For example, in the context of certain embodiments of the methods of the present invention, an effective amount of an antithrombotic agent is that amount sufficient to reduce or ameliorate thrombosis or a related disease or disorder.

In certain embodiments, the methods of the present invention are based upon the surprising discovery that previous assays of antithrombotic agent activity significantly underestimated that amount of antithrombotic agent necessary for effective inhibition of platelet aggregation in vivo. Accordingly, particular methods of the present invention are practiced using higher dosages or higher blood plasma concentrations of antithrombotic agent than previously thought necessary or previously used to treat patients. These may be, e.g., at least two-fold, at least three-fold, at least four-fold, at least five-fold, at least six-fold, at least seven-fold, at least eight-fold, at least nine-fold, or at least ten-fold higher than concentrations determined to be effective at inhibiting platelet aggregation using in vitro U-46619-induced platelet aggregation assays.

In particular embodiments, methods of the present invention comprise administering to a patient an amount of an antithrombotic agent sufficient to achieve a blood plasma concentration level the same as or comparable to the concentration shown to be effective in inhibiting platelet aggregation using an in vitro assay utilizing a physiological platelet agonist, as described herein. In particular embodiments, the blood plasma concentration is between 50% to 200% of the concentration shown to be effective in the in vitro assay. In particular embodiments, a concentration shown to be effective in an in vitro assay is a concentration equal to or greater than the concentration required to inhibit platelet aggregation to the same degree as aspirin usage. In another embodiment, it is the lowest concentration shown to inhibit at least 25% of platelet aggregation. In yet another embodiment, it is the concentration required to achieve at least 70%, at least 80%, at least 90% or 100% of the maximum inhibition of platelet aggregation achieved in an in vitro assay described herein.

In various embodiments, methods of the present invention comprise administering to a patient an amount of an antithrombotic agent sufficient to maintain a blood plasma concentration level the same as or comparable to the concentration shown to be effective in inhibiting platelet aggregation using an in vitro assay utilizing a physiological platelet agonist, as described herein, for at least six hours, at least 12 hours, at least 24 hours, at least 48 hours, or at least 72 hours. In particular embodiments, the blood plasma concentration is between 50% to 200% of the concentration shown to be effective in the in vitro assay. The amount of antithrombotic agent may be administered as a single dose, or in may be administered periodically to maintain the desired blood plasma concentration. For example, an antithrombotic agent may be administered every 6, 12, 24, 48, or 72 hours for a period of time.

In one embodiment, the present invention provides a method of reducing or inhibiting platelet aggregation or thrombosis, comprising providing to a patient an amount of an antithrombotic agent sufficient to achieve a blood plasma concentration of the antithrombotic agent of at least 50 nM, at least 100 nM, at least 150 nM, at least 200 nM, at least 250 nM, at least 300 nM, at least 350 nM, at least 400 nM, at least 450 nM, at least 500 nM, at least 600 nM, at least 700 nM, at least 800 nM, at least 900 nM, or at least 1000 nM for some period of time. The period of time may be, e.g., at least 2 hours, at least 4 hours, at least 8 hours, at least 12 hours, at least 18 hours, at least 24 hours, or at least 48 hours. In another embodiment, the period of time may a time period related to chronic use of the antithrombotic agent, e.g., at least 3 months, at least 6 months, at least 9 months, at least one year, or longer.

In one particular embodiment, platelet aggregation or thrombosis is reduced or inhibited by providing to a patient an amount of ifetroban sufficient to achieve a blood plasma concentration of at least 350 nM for at least 12 hours or at least 24 hours. In a particular embodiment, the patient is provided with at least 450 mg of ifetroban to achieve a blood plasma steady-state concentration of at least 350 nM for at least 24 hours.

In another embodiment, the present invention includes a method of treating or preventing thrombosis, comprising administering an amount of ifetroban sufficient to achieve a total blood plasma concentration having a steady state concentration in the range of 350 nM to 1000 nM for some time.

The present invention also includes a method of treating or preventing thrombosis, comprising administering to a patient an amount of ifetroban sufficient to achieve a total blood plasma concentration having a Cmax of between 1500 to 2500 ng/mL. In another embodiment, the Cmax is 2188 ng/mL or less.

In another embodiment, the present invention includes a method of treating or preventing thrombosis, comprising administering to a patient an amount of ifetroban sufficient to achieve a total blood plasma concentration having a mean trough concentration in the range of 100 to 200 ng/mL. In one embodiment, the mean trough concentration is 154 ng/mL.

In another embodiment, the present invention includes a method of treating or preventing thrombosis, comprising administering to a patient an amount of ifetroban sufficient to achieve a total blood plasma concentration having a peak to trough concentration ratio of 15 o less.

In particular embodiments, an antithrombotic agent is administered in an amount sufficient to achieve a blood plasma concentration greater than or equal to 1, 5, 10, 20, 50, 100, 150, 200, 250, 300, 350, 400, 450, 500, 550, 600, 650, 700, 750, 800, 850, 900, 950, or 1000 nM. In particular embodiments, it is administered in an amount sufficient to achieve a blood plasma level greater than or equal to 250 nM or 350 nM. In certain embodiments, it is administered in an amount sufficient to achieve a blood plasma concentration in the range of 1-10 nM, 1-100 nM, 10-1000 nM, 50-500 nM, 100-500 nM, 200-400 nM, 200-1000 nM, or 500-1000 nM.

In particular embodiments, ifetroban is administered in an amount sufficient to achieve a blood plasma concentration of at least 100 nM, at least 150 nM, at least 200 nM, at least 250 nM, at least 300 nM, at least 350 nM, at least 400 nM, at least 450 nM, at least 500 nM, at least 550 nM, or greater than 550 nM. In certain embodiments, ifetroban is administered in an amount sufficient to maintain a blood concentration of at least 100 nM, at least 150 nM, at least 200 nM, at least 250 nM, at least 300 nM, at least 350 nM, at least 400 nM, at least 450 nM, at least 500 nM, at least 550 nM, or greater than 550 nM for at least 6, 12, 24, or 48 hours.

In particular embodiments, an agent is administered in an amount within the range of from about 0.01 mg/kg to about 100 mg/kg, from about 0.1 mg/kg to about 100 mg/kg, from about 1 mg/kg to about 100 mg/kg, or from about 10 mg/kg to about 100 mg/kg. In particular embodiments, an antithrombotic agent is administered in an amount within the range of about 1 mg/kg to about 10 mg/kg, from about 2 mg/kg to about 10 mg/kg, from about 4 mg/kg to about 8 mg/kg or about 6 mg/kg to about 8 mg/kg. In one embodiment, it is administered at approximately 7 mg/kg. Thus, in particular embodiments, a total amount of between approximately 100-1000 mg, 100-500 mg, 200-500 mg, 300-500 mg, or 400-500 mg is administered to a patient as a dose. In one embodiment, approximately 450 mg is administered to a patient. In particular embodiments, administrating is oral or intravenous administration.

Additional methods of the present invention involve treating or preventing thrombosis or a cardiac disease or disorder by administering an antithrombotic agent in an amount described herein in combination with one or more additional therapeutic agents. In one embodiment, the additional therapeutic agent is an ADP receptor blocking antiplatelet drug, including but not limited to any of those described herein. Examples of such drugs include clopidogrel, ticlopidine, and 2-acetoxy-5-(α-cyclopropylcarboxyl-2-fluorobenzyl)-4,5,6,7-tetrahydrothieno[3,2-c]pyridine. In another embodiment, the additional therapeutic agent is an inhibitor of Factor Xa.

### C. Pharmaceutical Compositions and Unit Dose Formulations of Antithrombotic Agents

Antithrombotic agents and other therapeutic agents may be administered to a patient in pharmaceutical compositions via various routes of delivery, including e.g., oral, parenteral, intravenous, intranasal, and intramuscular administration. These and other routes of administration and suitable pharmaceutical formulations are well known in the art, some of which are briefly discussed below for general purposes of illustration. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. Pharmaceutical compositions of the invention are generally formulated so as to allow the active ingredients contained therein to be bioavailable upon administration of the composition to a patient.

It will be apparent that any of the compositions described herein can contain pharmaceutically acceptable salts of the antithrombotic agents of the invention. Such salts can be prepared, for example, from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

Therefore, in one aspect of the present invention, pharmaceutical compositions are provided comprising one or more of the antithrombotic agents described herein in combination with a physiologically or pharmaceutically acceptable diluent, excipient, or carrier. “Pharmaceutically acceptable carriers” for therapeutic use are well known in the pharmaceutical art, and are described, for example, in Remingtons Pharmaceutical Sciences, Mack Publishing Co. (A. R. Gennaro edit. 1985). For example, sterile saline and phosphate-buffered saline at physiological pH may be used. Preservatives, stabilizers, dyes and even flavoring agents may be provided in the pharmaceutical composition. For example, sodium benzoate, sorbic acid and esters of p-hydroxybenzoic acid may be added as preservatives. Id. at 1449. In addition, antioxidants and suspending agents may be used. Id.

While any suitable carrier known to those of ordinary skill in the art may be employed in the compositions of this invention, the type of carrier will typically vary depending on the mode of administration. Compositions of the present invention may be formulated for any appropriate manner of administration, including for example, topical, oral, nasal, mucosal, intravenous, intracranial, intraperitoneal, subcutaneous and intramuscular administration. In certain circumstances it will be desirable to deliver the antithrombotic agents disclosed herein parenterally, intravenously, intramuscularly, or even intraperitoneally. The term parenteral as used herein includes subcutaneous injections, intravenous, intramuscular, epidural, intrasternal injection or infusion techniques. Such approaches are well known to the skilled artisan, some of which are further described, for example, in U.S. Pat. No. 5,543,158; U.S. Pat. No. 5,641,515 and U.S. Pat. No. 5,399,363.

In certain embodiments, solutions of the agents as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations generally will contain a preservative to prevent the growth of microorganisms.

Illustrative pharmaceutical forms suitable for injectable use include sterile aqueous solutions or dispersions and sterile powders for the extemporaneous preparation of sterile injectable solutions or dispersions (for example, see U.S. Pat. No. 5,466,468). In one embodiment, for parenteral administration in an aqueous solution, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. Moreover, for human administration, preparations will of course preferably meet sterility, pyrogenicity, and the general safety and purity standards as required by FDA Office of Biologics standards.

The carriers can further comprise any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions. The phrase “pharmaceutically-acceptable” refers to molecular entities and compositions that do not produce an allergic or similar untoward reaction when administered to a human.

In certain embodiments, a pharmaceutical composition comprises one or more pharmaceutically acceptable carriers or diluents, buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate.

The compositions described herein may be presented in unit-dose or multi-dose containers, such as sealed ampoules or vials. Such containers are typically sealed in such a way to preserve the sterility and stability of the formulation until use. In particular embodiments, formulations may be stored as suspensions, solutions or emulsions in oily or aqueous vehicles. Alternatively, a composition may be stored in a freeze-dried condition requiring only the addition of a sterile liquid carrier immediately prior to use.

In certain applications, the antithrombotic agents disclosed herein may be delivered via oral administration to an animal. As such, these compositions may be, e.g., formulated with an inert diluent or with an assimilable edible carrier, or they may be enclosed in hard- or soft-shell gelatin capsule, or they may be compressed into tablets, or they may be incorporated directly with the food of the diet.

Compositions for administration to a patient may take the form of one or more dosage units, where for example, a tablet, capsule or cachet may be a single dosage unit, or a container of ion channel modulating compound in aerosol form may hold a plurality of dosage units. In particular embodiments, a composition comprising an antithrombotic agent, such as a TP antagonist, is administered in one or more doses of a tablet formulation, typically for oral administration. The tablet formulation may be, e.g., an immediate release formulation, a controlled release formulation, or an extended release formulation. In particular embodiments, a tablet comprises about 1, 5, 10, 20, 30, 50, 100, 125, 150, 175, 200, 225, 250, 275, 300, 350, 400, 450, 500, 550, 600, 650, 700, 750, 800, 850, 900, 950, or 1000 mg of an antithrombotic agent, or a TP antagonist, such as ifetroban. In particular embodiments, a tablet formulation comprises about 200-250 or 400-500 mg of ifetroban.

As used herein, “controlled release” refers to the release of the active ingredient from the formulation in a sustained and regulated manner over a longer period of time than an immediate release formulation containing the same amount of the active ingredient would release during the same time period. For example, an immediate release formulation comprising an antithrombotic agent may release 80% of the active ingredient from the formulation within 15 minutes of administration to a human subject, whereas an extended release formulation of the invention comprising the same amount of an antithrombotic agent would release 80% of the active ingredient within a period of time longer than 15 minutes, preferably within 6 to 12 hours. Controlled release formulations allows for less frequency of dosing to the mammal in need thereof. In addition, controlled release formulations may improve the pharmacokinetic or toxicity profile of the compound upon administration to the mammal in need thereof.

As used herein, “extended release” refers to the release of the active ingredient from the formulation in a sustained and regulated manner over a longer period of time than an immediate release formulation containing the same amount of the active ingredient would release during the same time period. For example, an immediate release formulation comprising an antithrombotic agent may release 80% of the active ingredient from the formulation within 15 minutes of administration to a human subject, whereas an extended release formulation of the invention comprising the same amount of antithrombotic agent would release 80% of the active ingredient within a period of time longer than 15 minutes, preferably within a period of time longer than 12 hours, e.g., 24 hours. Furthermore, the extended release formulations of the invention release the active ingredient, preferably ifetroban, over a longer period of time in vivo than a comparative controlled release formulation containing the same amount of the active ingredient would over the same period of time. As a non-limiting example, a comparative controlled release formulation containing the active ingredient, ifetroban, may release 80% of the amount of the active ingredient present in the formulation in vivo over a period of 4-6 hours after administration to a human subject, whereas an extended release formulation of the invention may release 80% of the same amount of the active ingredient in vivo over a period of 6-24 hours. Extended release formulations of the invention therefore allow for less frequency of dosing to the patient than the corresponding controlled release formulations. In addition, extended release formulations may improve the pharmacokinetic or toxicity profile of the active ingredient upon administration to the patient.

The present invention further includes unit dosage forms of pharmaceutical compositions comprising an antithrombotic agent. Each unit dosage form comprises a therapeutically effective amount of a pharmaceutical composition of the present invention, when used in the recommended amount. For example, a unit dosage form may include a therapeutically effective amount in a single tablet, or a unit dosage form may include a therapeutically effective amount in two or more tablets, such that the prescribed amount comprises a therapeutically effective amount.

The present invention provides unit dosage forms of antithrombotic agents, suitable for administering the agents at therapeutically effective dosages, e.g., dosages sufficient to achieve a blood plasma concentration described herein. These unit dose formulations may be prepared for administration to a patient once a day, twice a day, or more than twice a day. The desired dose of the pharmaceutical composition according to this invention may conveniently be presented in a single dose or as divided dose administered at appropriate intervals, for example as two, three or more doses per day. In certain embodiments, a suitable daily dose for an adult is between 1 and 5000 mg, between 1 and 1000 mg, between 10 and 1000 mg, between 50 and 500 mg, between 100 and 500 mg, between 200 and 500 mg, between 300 and 500 mg, or between 400 and 500 mg per day. Accordingly, when administered twice daily, a suitable single dose for an adult is between 0.5 and 2500 mg, between 0.5 and 500 mg, between 5 and 500 mg, between 25 and 250 mg, between 50 and 250 mg, between 100 and 250 mg, between 150 and 250 mg, or between 200 and 250 mg. Unit dose formulations may be readily adapted for multi-dosing.

In particular embodiments, a unit dosage form of ifetroban is a single capsule containing about 450 mg of ifetroban, or two capsules, each containing about 225 mg of ifetroban.

As described herein, an antithrombotic agent of the present invention may be used in combination with one or more other antithrombotic agents or pharmaceutical agents, including, e.g., a TP antagonist, a thrombixane antagonist, an ADP receptor antagonist, or a Factor Xa antagonist. When used in combination, it is understood that lower dosages of one or more of the combined antithrombotic agents may be utilized to achieve a desired effect, since the two or more antithrombotic agents may act additively or synergistically. Accordingly, a therapeutically effective dosage of one or more combined antithrombotic agents may correspond to less than 90%, less than 80%, less than 70%, less than 60%, less than 50%, less than 40%, less than 30% or less than 20% of the therapeutically effective dosage when the antithrombotic agent is administered alone.

The two or more antithrombotic agents may be administered at the same time or at different times, by the same route of administration or by different routes of administration. For example, in order to regulate the dosage schedule, the antithrombotic agents may be administered separately in individual dosage units at the same time or different coordinated times. The respective substances can be individually formulated in separate unit dosage forms in a manner similar to that described above. However, fixed combinations of the antithrombotic agents are more convenient and are preferred, especially in tablet or capsule form for oral administration.

Thus, the present invention also provides unit dose formulations comprising two or more antithrombotic agents, wherein each thrombotic agent is present in a therapeutically effective amount when administered in the combination.

In particular embodiments, a patient is provided with ifetroban and one or more additional antithrombotic agents. In addition, the present invention includes a combination unit dose formulation comprises ifetroban and one or more addition antithrombotic agents. For example, methods of the present invention may comprise providing to a patient ifetroban in combination with another TP antagonist or an ADP receptor antagonist. In particular embodiments, ifetroban is provided in combination with a P2Y12 inhibitor, clopidogrel, prasugrel, or cangrelor. In particular embodiments, additional antithrombotic agents are provided (in combination with ifetroban or another antithrombotic agent) in an amount previously indicated as effective when the agent is used in combination with aspirin.

In certain embodiments, clopidogrel is provided in an oral daily dosage within the range from about 10 to about 1000 mg and preferably from about 25 to about 600 mg, and most preferably from about 50 to about 100 mg. In one particular embodiment, approximately 400-500 mg of ifetroban and approximately 50-100 mg of clopidogrel is provided to a patient per day. In a related embodiment, approximately 200-400 mg of ifetroban and 25-50 mg of clopidogrel is provided to a patient per day. In one particular embodiment, a patient is provided with about 450 mg of ifetroban and about 75 mg of clopiogrel (e.g., Plavix®) per day.

In certain embodiments, ticlopidine is provided in a daily dosage as set out in the 1997 PDR (250 mg bid) although daily dosages of from about 10 to about 1000 mg, preferably from about 25 to about 800 mg may be employed in accordance with the present invention. In one particular embodiment, approximately 400-500 mg of ifetroban and approximately 250-750 mg of ticlopidine is provided to a patient per day. In a related embodiment, approximately 200-400 mg of ifetroban and 100-250 mg of ticlopidine is provided to a patient per day. In one particular embodiment, a patient is provided with about 450 mg of ifetroban and about 500 mg of ticlopidine (e.g., Tidclid®) per day.

In certain embodiments, prasugrel is provided in a daily dosage of 1 to 100 mg per day, or about 10 mg per day. In one particular embodiment, approximately 400-500 mg of ifetroban and approximately 1 to 100 mg of prasugrel is provided to a patient per day. In a related embodiment, approximately 200-400 mg of ifetroban and 1 to 5 mg of prasugrel is provided to a patient per day. In one particular embodiment, a patient is provided with about 450 mg of ifetroban and about 10 mg of prasugrel per day.

The present invention further provides unit dosages comprising ifetroban and one or more additional antithrombotic agents, including any of those described herein. In particular embodiments, the additional antithrombotic agent is an ADP receptor antagonist. In particular embodiments, the additional antithrombotic agent is a P2Y12 inhibitor. Unit dosages of the present invention, in particular embodiments, comprise a daily dosage of ifetroban and a daily dosage of the additional one or more antithrombotic agents. Alternatively, a unit dosage comprises a portion of a daily dosage such as 50% of a daily dosage of the antithrombitic agents, so that the daily dosage may be taken in two unit dosages, e.g., at the same time or at different times.

### EXAMPLES

### Example 1

### Identification of Therapeutically Effective Ifetroban Dosages

The concentration of ifetroban required to equate the antithrombotic activity of aspirin was determined using collagen-induced platelet aggregation and real-time perfusion chamber assays on anticoagulated (with an anticoagulant that does not affect physiological concentrations of calcium in plasma) samples of platelet-rich plasma (PRP) and blood, respectively. These assays indicated that concentrations of ifetroban that provide similar levels of inhibition of thrombosis as low doses aspirin (75-325 mg/d) in collagen-induced platelet aggregation assays are approximately 10 times higher than those predicted by the use of U-46619-induced platelet aggregation assays (350 nM v. 30 nM, respectively).

The platelet aggregation inhibitory activities of aspirin and ifetroban were first compared by light transmittance aggregometry (LTA) using samples of PRP anticoagulated with a Factor Xa inhibitor that does not affect physiological calcium concentration. Blood from healthy individuals (n=6-7) was obtained by venipuncture and anticoagulated with FXa inhibitor. Platelet rich plasma (PRP) was obtained, and LTA was performed by standard procedures, initiating platelet aggregation with U-46619 (10 μM) or collagen (4 μg/ml).

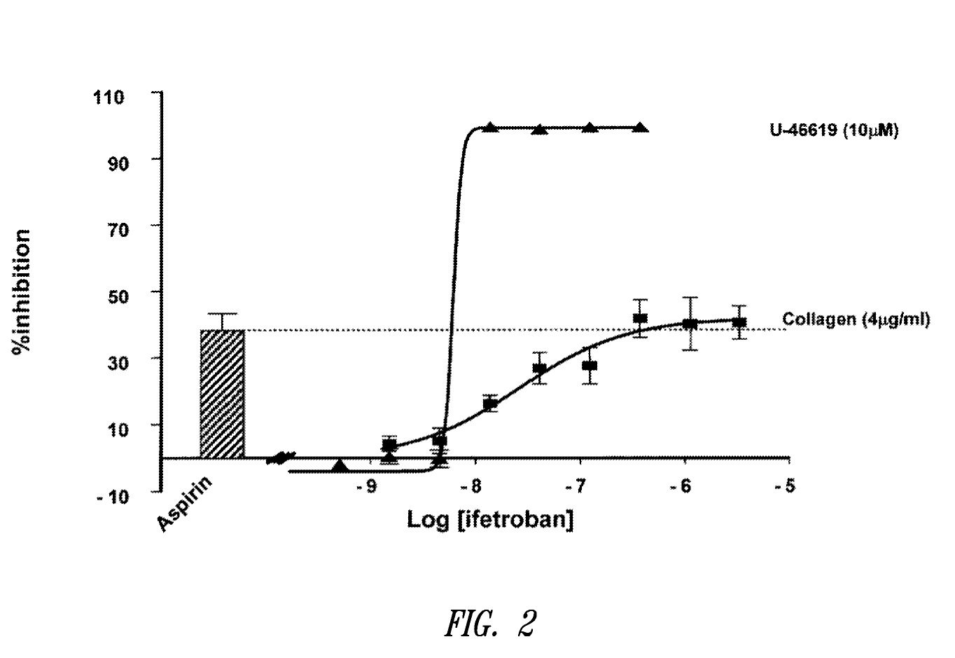

To establish the platelet aggregation inhibitory activity of aspirin, twenty healthy individuals were studied, both before and after two weeks of a daily regimen of aspirin (325 mg/day). LTA was performed on PRP samples using collagen (4 micrograms/ml) to induce platelet aggregation. As shown in FIG. 2, aspirin reduced the extent of collagen-induced platelet aggregation by 39+/−6.0% v. baseline (mean+sem).

The concentration of ifetroban required to achieve the inhibitory activity of aspirin was determined by adding increasing concentrations of ifetroban to non-aspirinated PRP in vitro, and performing LTA in the presence of U-46619 or collagen. The data presented in FIG. 2 demonstrates that ifetroban fully inhibited U-46619-induced platelet aggregation at concentrations >30 nM. On the other hand, maximum inhibition of collagen-induced platelet aggregation occurred at concentrations greater than or equal to 350 nM. These data indicate that approximately 350 nM ifetroban is required to inhibit platelet aggregation to a similar extent as aspirin (denoted by the dotted line in FIG. 2).

In another assay, the antithrombotic activities of aspirin and ifetroban were compared using a real time perfusion chamber assay, in which blood anticoagulated with Fxa inhibitor was perfused through collagen-coated capillaries. The antithrombotic activity of aspirin was determined using blood obtained from twenty normal subjects treated with 325 mg aspirin per day for 2 weeks.

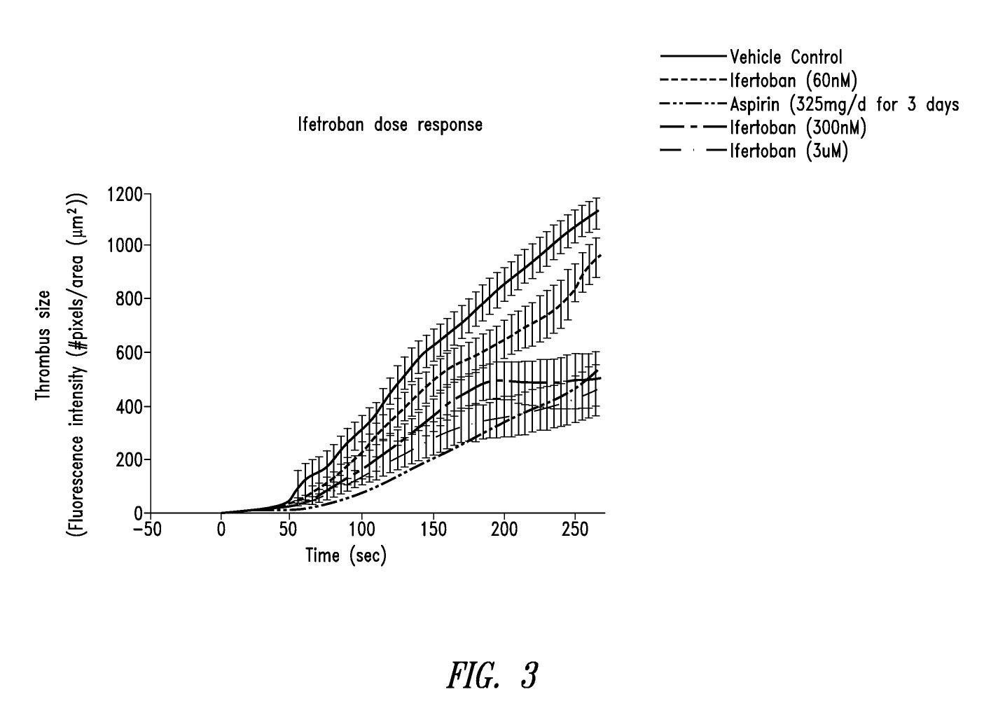

In order to determine the concentration of ifetroban that achieves the antithrombotic activity provided by aspirin, increasing concentrations of ifetroban were added to samples of non-aspirinated blood prior to perfusion through the chamber (n=6 different individuals). As shown in FIG. 3, ifetroban concentrations ≧300 nM provided equivalent or superior inhibitory activity on thrombosis as aspirin.

These data demonstrate that the amount of ifetroban required to inhibit platelet aggregation induced by collagen as compared to platelet aggregation induced by U-46619 are substantially higher (approximately 10-fold). Accordingly, it can be predicted that therapeutically effective dosages of ifetroban are much higher than those previously determined using U-46619-induced aggregation assays. In addition, these experiments demonstrate that collagen-induced platelet aggregation assays, which are more biologically relevant than U-46619-induced platelet aggregation assays, may be used to determine therapeutically effective dosages of antithrombotic agents, including TP antagonists such as ifetroban.

### Example 2

### Antiplatelet Effects of Ifetroban in Aspirin-Tolerant and Aspirin-Sensitive Patients

The thrombotic profile of aspirin intolerant (AERD)-asthmatic patients (AIA) patients and healthy volunteers was evaluated by comparing the antiplatelet effects of PRT061103 and aspirin after desensitization using a physiological platelet agonist, essentially as described in Example 1.

Real time perfusion chamber assays (RTTP) were performed using blood anticoagulated with Fxa inhibitor (10 uM 034) and perfused through collagen-coated capillaries (1100/sec). Thrombus formation on the collagen surface was monitored in real time using fluorescence microscopy to detect fluorescently labeled (R6G) platelets.

Light transmittance aggregometry (LTA) assays were performed by standard procedures, initiating platelet aggregation with collagen or arachidonic acid.

Assays were performed pre- (+/−ifetroban, spiked in vitro) and post-aspirin desensitization.

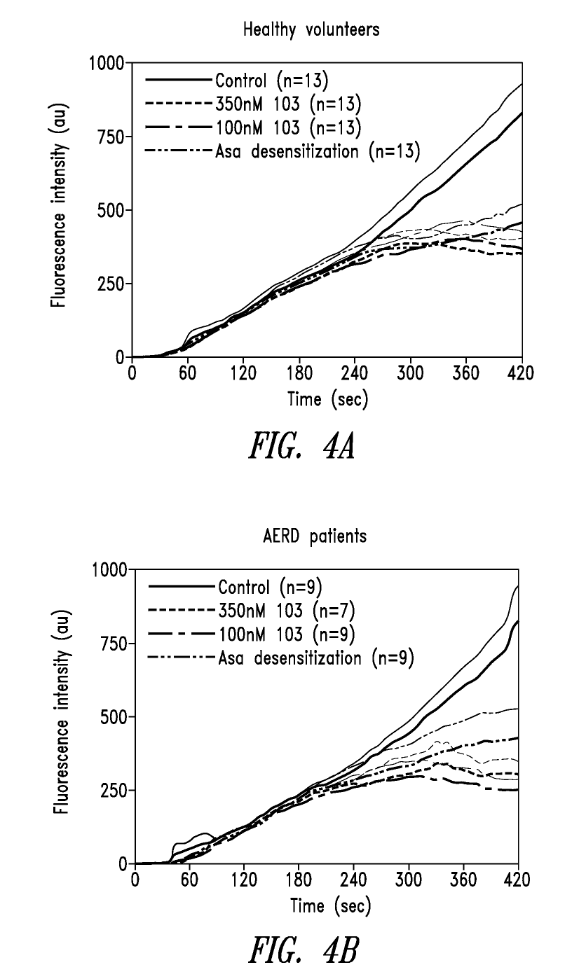

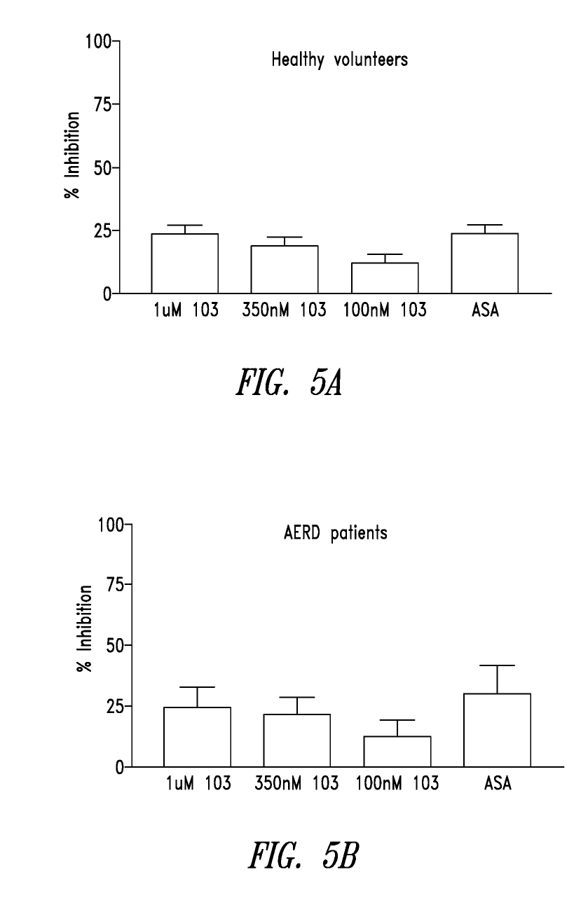

As shown in FIG. 4, PRT061103 had significant antithrombotic activity in both healthy volunteers (FIG. 4A) and AERD patients (FIG. 4B) when measured using the perfusion chamber assay. Ifetroban also showed significant anti-aggregatory activity in both healthy volunteers and AERD patients in the collagen-induced platelet aggregation assay (FIG. 5). Specifically, PRT061103 reproduced aspirin effects on collagen-induced platelet aggregation and thrombosis at concentrations >100 nM in both normal volunteers (FIG. 5A) and AERD patients (FIG. 5B). In healthy volunteers, 100 nM ifetroban had a significantly lower inhibition of platelet aggregation than aspirin, while 350 and 1000 nM ifetroban displayed similar levels of inhibition as aspirin.

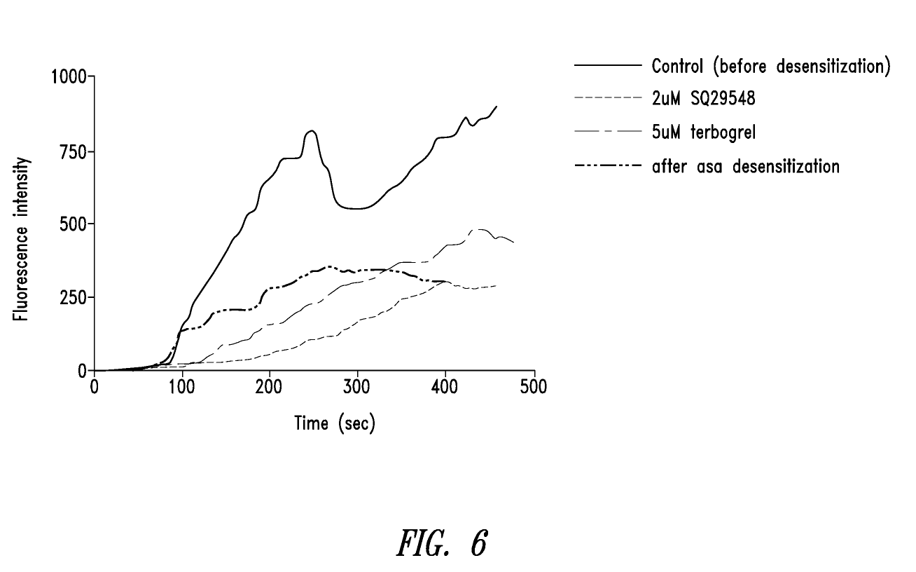

The ability of other TP antagonists to inhibit thrombosis was demonstrated using SQ29548, a direct acting TP antagonist, and terbogrel, a mixed inhibitor of TP and TxA synthase. As determined using the perfusion chamber test (RTTP), both SQ29548 and terbogrel spiked in vitro provided similar levels of inhibition of thrombosis as aspirin (after desensitization) in AERD patients (FIG. 6).

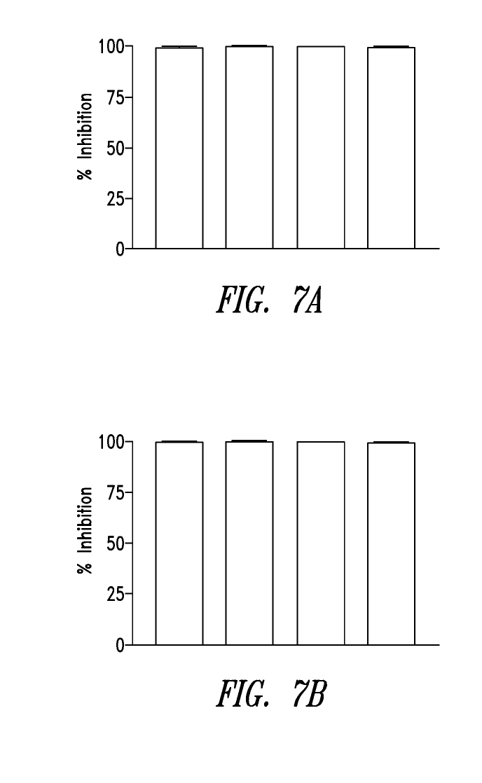

The anti-aggregatory activity of PRT061103 versus aspirin in healthy volunteers and AERD patients was further demonstrated using an arachidonic acid-induced platelet aggregation assay (FIGS. 7A and 7B). Here, all three tested doses of ifetroban fully blocked arachidonic acid-induced platelet aggregation. However, arachidonic acid is not a physiological platelet agonist, so this assay does not define a dose that provides effective concentration in vivo.

These data demonstrate that platelet aggregation assays performed using physiological platelet agonists, e.g., collagen or arachidonic acid, may be used to determine therapeutically effective dosages of antithrombotic agents, including TP antagonists such as ifetroban, in both aspirin tolerant and aspirin-intolerant or aspirin-sensitive patients.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for inhibiting platelet aggregation in a patient in need thereof, comprising administering to a patient in need thereof a therapeutic concentration of an antithrombotic agent, wherein said therapeutic concentration is determined by a method comprising:
(a) contacting a blood sample obtained from a mammal with a physiological platelet agonist in an amount sufficient to induce platelet aggregation in the blood sample and measuring a first amount of platelet aggregation; and
(b) subsequently contacting the blood sample with a plasma concentration of an antithrombotic agent and measuring a second amount of platelet aggregation in the blood sample, wherein if the second amount of platelet aggregation is at least 25% lower than the first amount of platelet aggregation, the plasma concentration of the antithrombotic agent is a therapeutically effective plasma concentration.

2. The method of claim 1, wherein the physiological platelet agonist is collagen.

3. The method of claim 1, further comprising anticoagulating the blood sample prior to contacting the blood sample with the physiological platelet agonist.

4. The method of claim 1, wherein the antithrombotic agent is a thromboxane receptor antagonist.

5. The method of claim 4, wherein the thromboxane receptor antagonist is ifetroban.

6. The method of claim 1, wherein the first amount and second amount of platelet aggregation is measured by light transmittance aggregometry.

7. The method of claim 2, wherein the first amount and second amount of platelet aggregation is measured using a real time perfusion chamber.

8. A method of inhibiting aggregation of platelets, comprising contacting platelets with ifetroban at a concentration greater than 100 nM.

9. A method of treating or preventing thrombosis in a patient, comprising administering to the patient an amount of ifetroban sufficient to achieve a plasma concentration greater than 100 nM for at least 24 hours.

10. The method of claim 9, wherein the amount of ifetroban is between 1 and 10 mg/kg/day.

11. The method of claim 9, wherein the amount is sufficient to achieve a plasma concentration greater than or equal to 350 nM for at least 12 hours.

12. The method of claim 9, wherein the amount is sufficient to achieve a plasma concentration greater than or equal to 350 nM for at least 24 hours.

13. The method of claim 12, wherein the amount of ifetroban is between 6 and 10 mg/kg/day.

14. A method of treating or preventing thrombosis in a patient, comprising administering a therapeutically effective plasma concentration of an antithrombotic agent to the patient, wherein the therapeutically effective plasma concentration is determined by a method comprising:
(a) contacting a blood sample obtained from a mammal with a physiological platelet agonist in an amount sufficient to induce platelet aggregation in the blood sample and measuring a first amount of platelet aggregation; and
(b) subsequently contacting the blood sample with a plasma concentration of an antithrombotic agent and measuring a second amount of platelet aggregation in the blood sample, wherein if the second amount of platelet aggregation is at least 25% lower than the first amount of platelet aggregation, the plasma concentration of the antithrombotic agent is a therapeutically effective plasma concentration.

15. The method of claim 14, wherein the physiological platelet agonist is collagen.

16. The method of claim 14, further comprising anticoagulating the blood sample prior to contacting the blood sample with the physiological platelet agonist.

17. The method of claim 14, wherein the antithrombotic agent is a thromboxane receptor antagonist.

18. The method of claim 17, wherein the thromboxane receptor antagonist is ifetroban.

19. The method of claim 14, wherein the first amount and second amount of platelet aggregation is measured by light transmittance aggregometry.

20. The method of claim 14, wherein the first amount and second amount of platelet aggregation is measured using a real time perfusion chamber.

21. A unit dose formulation of ifetroban, comprising a pharmaceutically acceptable carrier and an amount of ifetroban sufficient to maintain a plasma concentration of 250 nM for at least 24 hours.

22. The unit dose formulation of claim 21, wherein the amount of ifetroban is sufficient to maintain a plasma concentration of 350 nM for at least 24 hours.

23. The unit dose formulation of claim 21, wherein the formulation is adapted for once a day administration and the amount of ifetroban is a dose between 6-10 mg/kg.

24. The unit dose formulation of claim 21, wherein the formulation is adapted for twice a day administration and the amount of ifetroban is a dose between 3-5 mg/kg.

25. A method for determining an effective concentration of an antithrombotic agent for inhibiting aggregation of mammalian platelets, comprising:
(a) contacting a blood sample obtained from a mammal with a physiological platelet agonist in an amount sufficient to induce platelet aggregation in the blood sample and measuring a first amount of platelet aggregation; and
(b) subsequently contacting the blood sample with a plasma concentration of the antithrombotic agent and measuring a second amount of platelet aggregation in the blood sample, wherein, if the second amount of platelet aggregation is at least 25% lower than the first amount of platelet aggregation, the plasma concentration is an effective concentration of the antithrombotic agent for inhibiting aggregation of mammalian platelets.

26. The method of claim 25, wherein the physiological platelet agonist is selected from the group consisting of: collagen, epinephrine, and ADP.

27. The method of claim 25, further comprising anticoagulating the blood sample prior to contacting the blood sample with the physiological platelet agonist.

28. The method of claim 25, wherein the antithrombotic agent is a thromboxane receptor antagonist.

29. The method of claim 28, wherein the thromboxane receptor antagonist is ifetroban.

30. A method of treating or preventing thrombosis in a patient, comprising providing to a patient an amount of ifetroban sufficient to achieve a steady-state blood plasma concentration of at least 350 nM for some time.

31. The method of claim 31, wherein the amount of ifetroban is sufficient to achieve a steady-state blood plasma concentration in the range of 350 nM and 1000 nM for some time.

32. A method of treating or preventing thrombosis in a patient, comprising providing to a patient an amount of ifetroban sufficient to achieve a blood plasma concentration having a Cmax in the range of 1500 to 2500 ng/mL.

33. A method of treating or preventing thrombosis in a patient, comprising providing to a patient amount of ifetroban sufficient to achieve a total blood plasma concentration having a mean trough concentration of about 154 ng/mL.

34. A method of treating or preventing thrombosis in a patient, comprising administering to a patient an amount of ifetroban sufficient to achieve a total blood plasma concentration having a peak to trough concentration ratio of 15 or less.

35. A method of treating or preventing a cardiovascular disease or disorder in a patient, comprising providing to the patient ifetroban in an amount sufficient to achieve a blood plasma concentration greater than 100 nM for at least 12 hours.

36. The method of claim 35, wherein the plasma concentration is greater than 100 nM for at least 12 hours.

37. The method of claim 35, wherein the amount is sufficient to achieve a plasma concentration greater than or equal to 350 nM for at least 12 hours.

38. The method of claim 37, wherein the amount is sufficient to achieve a plasma concentration greater than or equal to 350 nM for at least 24 hours.

39. The method of claim 35, wherein the amount of ifetroban is between 1 and 10 mg/kg/day.

40. The method of claim 39, wherein the amount of ifetroban is between 6 and 10 mg/kg/day.

41. The method of claim 35, wherein the cardiovascular disease or disorder is selected from the group consisting of: myocardial infarction, thrombotic stroke, atherosclerotic disease, unstable angina, refractory angina, transient ischemic attacks, embolic stroke, disseminated intravascular coagulation, septic shock, deep venous thrombosis, pulmonary embolism, reocclusion, restenosis, pulmonary embolism, occlusive coronary thrombus, complications resulting from thrombolytic therapy, percutaneous transluminal coronary angioplasty, or coronary artery bypass grafts, pulmonary hypertension, and intravascular thrombosis.
